(19) United States
(12) Patent Application Publication
(10) Pub. No.: US 2025/0018135 A1
(43) Pub. Date: Jan. 16, 2025

(54) DEVICE FOR DISPENSING A FLUID PRODUCT

(71) Applicant: APTAR FRANCE SAS, Le Neubourg (FR)

(72) Inventor: Matthieu BAILLET, Rouen (FR)

(73) Assignee: APTAR FRANCE SAS, Le Neubourg (FR)

(21) Appl. No.: 18/712,909

(22) PCT Filed: Nov. 22, 2022
(86) PCT No.: PCT/FR2022/052150

§ 371 Date: May 23, 2024

(30) Foreign Application Priority Data
Nov. 24, 2021 (FR) FR2112457

(51) Int. Cl. A61M 15/08

(52) U.S. Cl. CPC A61M 15/08

(57) ABSTRACT

Device having a reservoir containing two doses of fluid product; a dispensing member; a main body; a central body; a support member accommodating the reservoir; a dispensing head; and an actuating member engaging the support member for successive actuations. The device has an energy accumulation mechanism with first and second pushing elements and first and second resistance elements. The first pushing element engages with the first resistance element to accumulate energy during the dispensing of the first dose, and the second pushing element engages with the second resistance element to accumulate energy during the dispensing of the second dose. The energy storage mechanism being produced such that the active parts of each pushing element do not make any contact with the support member during assembly, and the active part of the second pushing element does not make any contact with the support member during the dispensing of the first dose.

The present invention relates to a device for dispensing a fluid product of the bidose type, in particular for a nasal spray.

The term “dispenser device of the dual-dose type” means a device containing two doses of fluid to be dispensed during two successive actuations of the dispenser device.

Bidose type devices are well known in the prior art. Such devices generally comprise a reservoir containing the two doses of fluid to be dispensed, and a dispensing member that is generally a piston, that is mounted to slide in said reservoir, and that is moved so as to dispense the fluid contained in said reservoir. The piston is moved in two successive actuation strokes, such that a first dose is dispensed during a first actuation, and a second dose is dispensed during a second actuation.

With this type of device of the bidose type, there are dose separation means, to define the two doses, and generally also, energy accumulation means, provided to impose the application of a certain force to be able to actuate the device. These energy accumulation means make it possible to prevent undesired or accidental actuations, for example, during storage or transport. They also guarantee a dispensing of all of the dose upon each actuation, by generating a precompression in the user's hand at the time of actuation. These energy accumulation means, which can, for example, be formed by deformable movable or breakable elements, also make it possible to predefine the force necessary for the actuation, and therefore the parameters of the spray generated exiting from the device.

Yet, during the assembly and/or the actuation of the device, there is a risk of altering said energy accumulation means. Thus, during assembly, certain elements forming these energy accumulation means can undergo impacts which can modify them, consequently with a possible impact on the energy accumulation performance. A risk of increasing the variability of the actuation force required to overcome the energy accumulation means ensues, and therefore a variability in the properties of the spray during the dose dispensing. Yet, such a variability is not desirable, in particular in terms of repeatability and stability of dose dispensing performance. There is a similar risk during the actuation of the first dose for the energy accumulation means provided for the second dose, and which can, during this first actuation, undergo impacts which can alter them.

Documents US20100145275A1, US20160068326A1 and U.S. Pat. No. 7,681,570B2 describe devices of the prior art.

The object of this invention is to provide a device for dispensing a fluid product that does not have the above mentioned disadvantages.

The present invention thus aims to provide a device for dispensing a fluid product, which guarantees the integrity of the energy accumulation means before each use.

The present invention also aims to provide such a device for dispensing a fluid product, which reduces the variability of the actuation forces and improves the repeatability the stability of the dispensing parameters of each dose.

The present invention also aims to provide such a device for dispensing a fluid product, which is simple and inexpensive to manufacture and assemble.

The present invention therefore aims for a device for dispensing a fluid product comprising a reservoir containing two doses of fluid product, a dispensing member, such as a piston, slidingly mounted in said reservoir to dispense the fluid product, a main body, a central body secured to said main body, a support member that accommodates said reservoir, a dispensing head provided with a dispensing orifice and an actuating member that is axially displaceable within said main body and engages with said support member to carry out successive actuations of the device, by displacing said support member with respect to said central body to thus displace said actuating member in said reservoir, and thus dispense the fluid product through said dispensing orifice, a return spring being provided to return said actuating member into its starting position after each actuation, said device comprising energy accumulation means comprising first and second pushing elements formed on said central body and first and second resistance elements formed on said support member, said first pushing element being suitable for engaging with said first resistance element to accumulate energy during the dispensing of the first dose, and said second pushing element being suitable for engaging with said second resistance element to accumulate energy during the dispensing of the second dose, each pushing element comprising a radially deformable tab, provided at its lower axial end with a central pushing zone, the axially lower surface of which comes into contact with a respective resistance element during actuations, wherein:
said support member comprises two diametrically opposite longitudinal profiles, each longitudinal profile comprising, starting with the upper axial edge: a first deformation profile, a first window, a second deformation profile, and a second window,
the first window of a longitudinal profile comprises said resistance element, and the second window of the other longitudinal profile comprises said second resistance element,
each deformation profile comprises: a central ramp extending axially and inclined radially outwards towards the bottom, disposed between first and second axially straight side ridges and with a constant radial dimension,
each central pushing zone is disposed between a first and a second inclined zone,
such that when said support member is axially displaced with respect to said central body during assembly, each pushing element engages with a respective first deformation profile, such that each central pushing zone does not come into contact with said support member.

Advantageously, when said support member is axially displaced with respect to said central body during the dispensing of the first dose, said second pushing element engages with a second deformation profile, such that its central pushing zone does not come into contact with said support member.

Advantageously, said first and second resistance elements are axially offset on said support body.

Advantageously, said first and second pushing elements are disposed diametrically opposite on said central body.

Advantageously, said resistance elements are formed by breakable bridges, which are broken during actuation, or by deformable beams, which are deformed during actuation.

Advantageously, said first and second pushing elements are identical, said first pushing element engaging with a longitudinal profile and said second pushing element engaging with the other longitudinal profile.

Advantageously, said first deformation profiles engage with the two pushing elements during assembly, and said second deformation profiles engage with said second pushing elements during the dispensing of the first dose.

Advantageously, the radially internal surface of each pushing element is T-shaped.

Advantageously, during assembly, said pushing zones will each engage with a respective central ramp of a first deformation profile, while said inclined zones will engage with said side ridges.

Advantageously, during the dispensing of the first dose, said pushing zones will each engage with a respective central ramp of a second deformation profile, while said inclined zones will engage with said side ridges.

Advantageously, during assembly, said inclined zones of each pushing element slide over the upper axial edge of said side ridges of said first deformation profile, which deforms said deformable tabs radially outwards, such that each pushing zone avoids any contact with said support member, by first facing the part with the smallest diameter of each central ramp, then by moving away radially outwards according to the assembly.

Advantageously, during the dispensing of the first dose, said inclined zones of each pushing element slide over the upper axial edge of said side ridges of said second deformation profile, which deforms said deformable tabs radially outwards, such that each pushing zone avoids any contact with said support member, by first facing the part with the smallest diameter of each central ramp, then by moving away radially outwards according to the dispensing of the first dose.

Advantageously, at the end of assembly, said deformable tabs are snap-fitted in a respective first window, said first pushing element being disposed facing said first resistance element, to accumulate energy during the dispensing of the first dose.

Advantageously, at the end of dispensing the first dose, said deformable tabs are snap-fitted in a second respective window, said second pushing element being disposed facing said second resistance element, to accumulate energy during the dispensing of the second dose.

These advantages and characteristics of the present invention, and others, appear more clearly from the following detailed description, given by way of non-limiting examples, and with reference to the accompanying drawings, and in which:

FIGS. 1 to 4 are schematic, cross-sectional views of a device for dispensing a fluid product according to a first advantageous embodiment, respectively before dispensing of the first dose, after dispensing of the first dose, before dispensing of the second dose and after dispensing of the second dose,

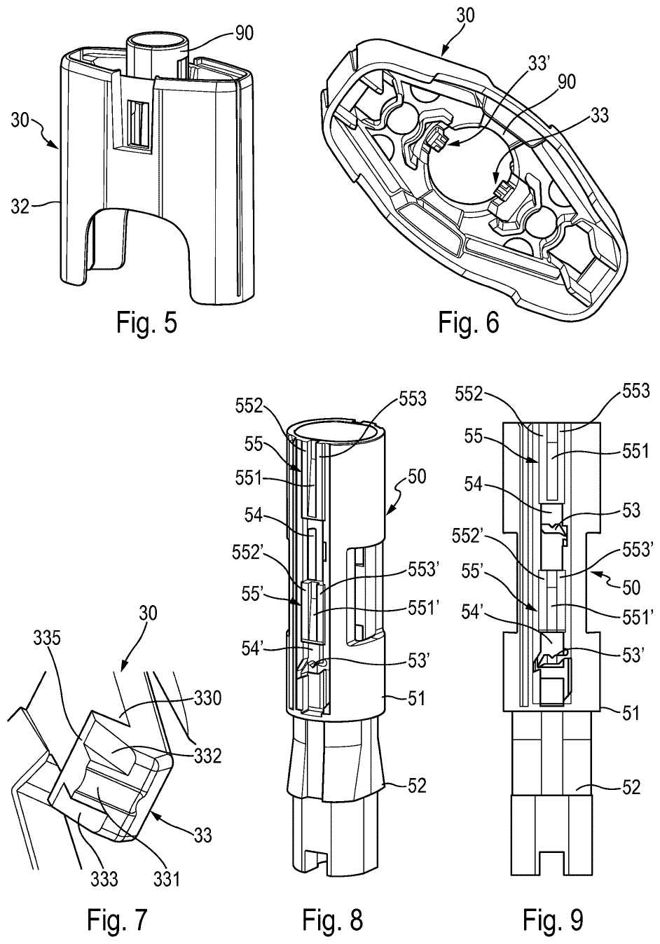

FIG. 5 is a schematic, perspective, side view of the main body of the device of FIGS. 1 to 4,

FIG. 6 is a schematic, perspective, bottom view of the main body of FIG. 5,

FIG. 7 is a schematic, partial, perspective view of a first pushing element secured to the main body of FIGS. 5 and 6,

FIGS. 8 and 9 are schematic, side views of the support body of the device of FIGS. 1 to 4,

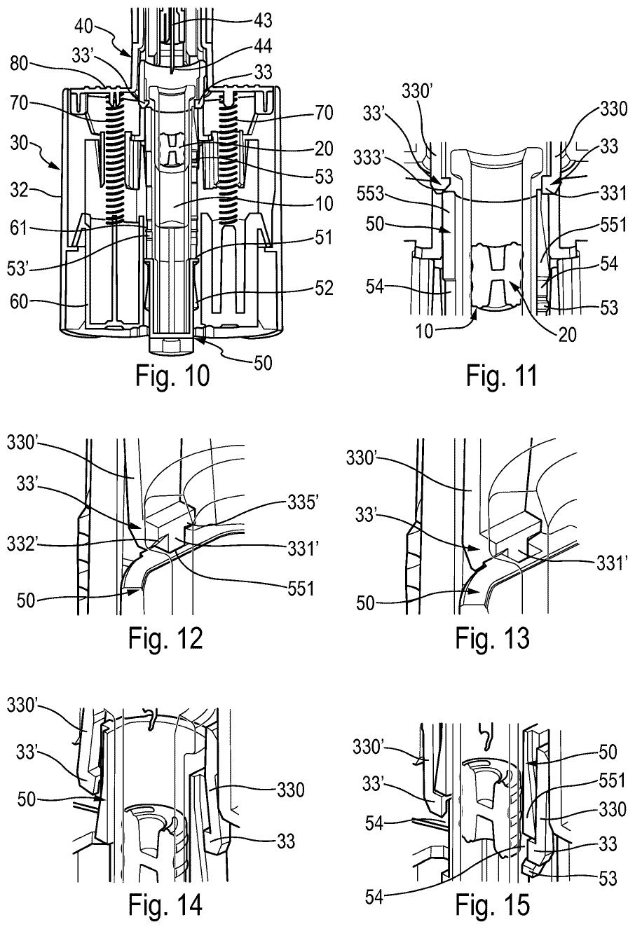

FIG. 10 is a schematic view during assembly of the support body in the main body,

FIG. 11 is a detailed, enlarged view of FIG. 10,

FIGS. 12 to 15 are enlarged, detailed views showing different positions of the pushing element during assembly,

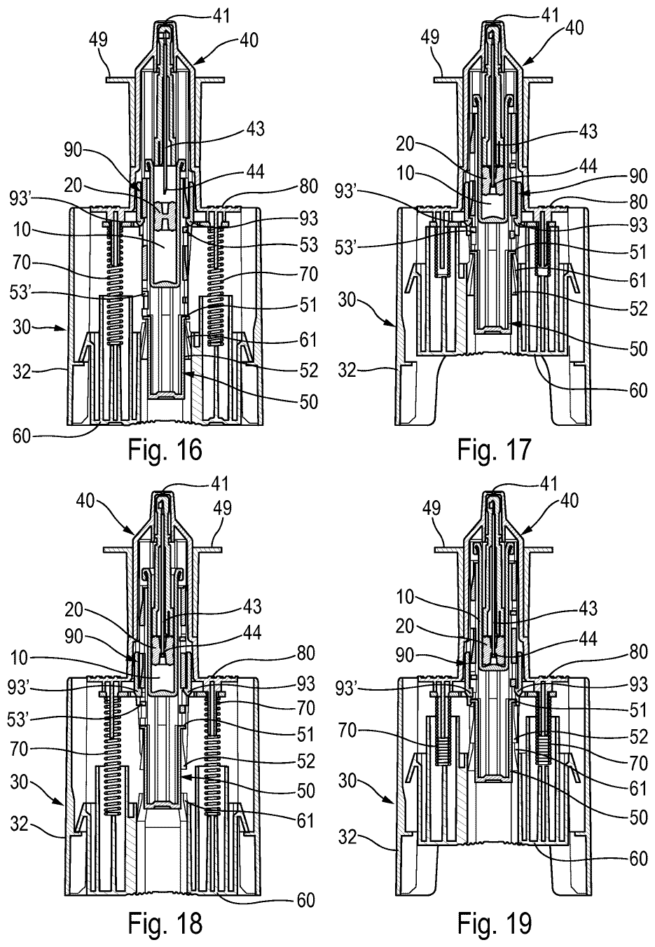

FIGS. 16 to 19 are schematic, cross-sectional view of a device for dispensing a fluid product according to a second advantageous embodiment, respectively before dispensing of the first dose, after dispensing of the first dose, before dispensing of the second dose and after dispensing of the second dose,

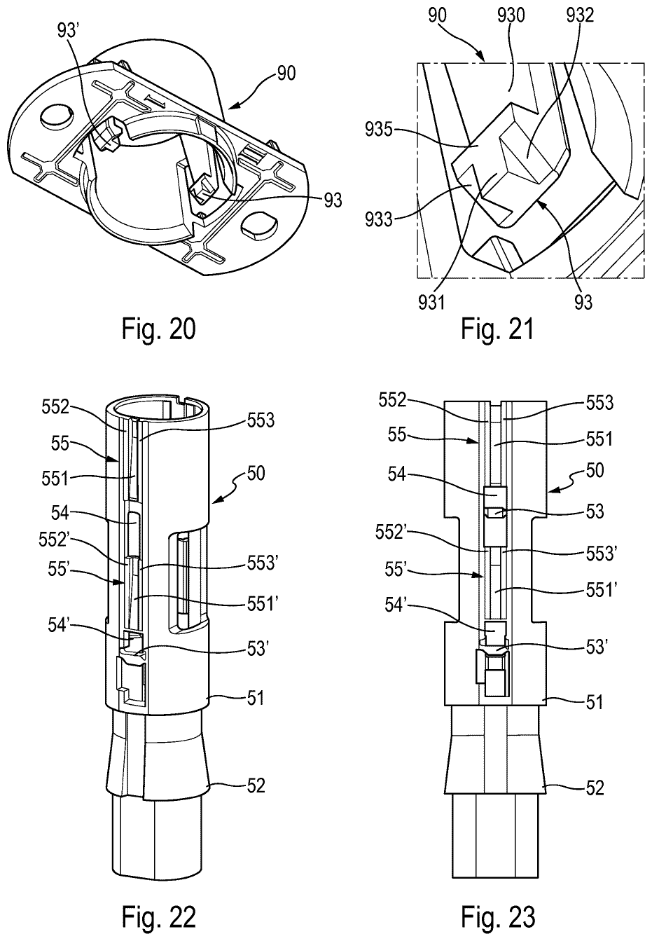

FIG. 20 is a schematic, perspective, bottom view of the central body of the device of FIGS. 1 to 4,

FIG. 21 is a schematic, partial, perspective view of a first pushing element secured to the main body of FIG. 20,

FIGS. 22 and 23 are schematic, side views of the support body of the device of FIGS. 16 to 19,

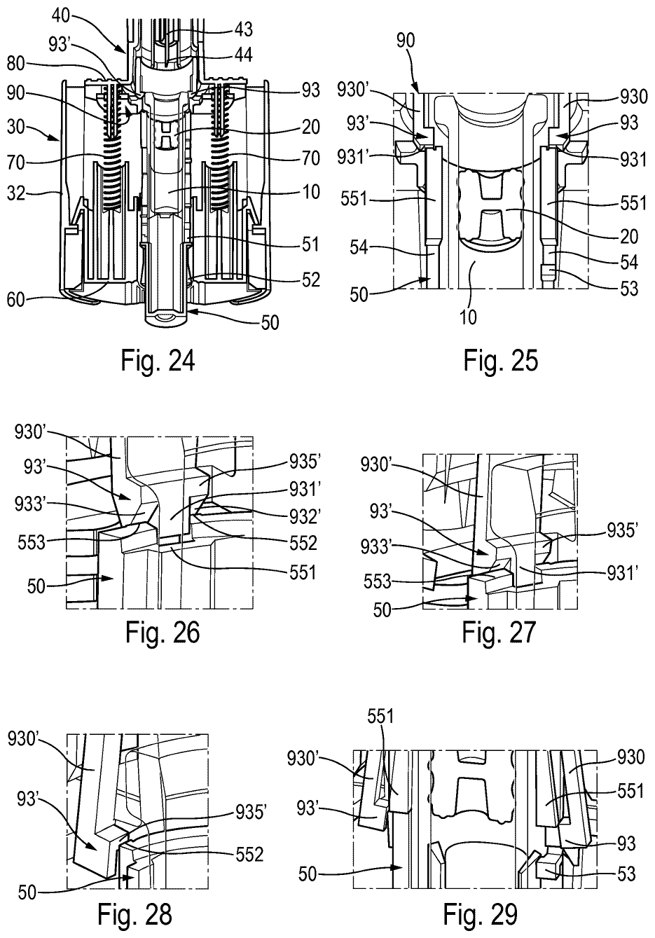

FIG. 24 is a schematic view during assembly of the support body in the central body,

FIG. 25 is a detailed, enlarged view of FIG. 24,

FIGS. 26 to 29 are enlarged, detailed views showing different positions of the pushing element during assembly.

In the description below, the terms “axial” and “radial” are relative to the longitudinal central axis of the device. The terms “top”, “bottom”, “upper” and “lower” refer to the straight position represented in FIGS. 1 to 4 and 16 to 19.

The present invention will be described below in reference to two embodiments of a bidose, i.e. a device containing two doses of fluid product to be dispensed during two successive actuations of the device. It is however understood that the bidose-type devices represented in the drawings are only examples of possible embodiments to which the present invention applies, and it is understood that the present invention applies more generally to any type of device containing two doses.

Referring to Figures, the dual-dose dispenser device includes a reservoir 10 containing two doses of fluid. A dispenser member, such as a piston 20, is mounted to slide in said reservoir 10. In the position before actuation of the device, represented in FIGS. 1 and 16, said piston 20 acts as a stopper by isolating the content from the reservoir 10.

A main body 30 is assembled on said reservoir 10 by being axially displaceable with respect to it. In particular, an axial displacement of the main body 30 with respect to the reservoir 10 causes the displacement of the piston 20 in the reservoir 10 and thus the dispensing of the fluid product contained in said reservoir.

A dispensing head 40 is assembled on the main body 30. The dispensing head 40 includes a dispensing channel 43 that extends from a perforator tip 44 to the dispensing orifice 41 of the dispensing head 40. A spray profile, which may be of any known type and not shown in more detail in the drawings, can be provided upstream of the dispensing orifice 41 for dispensing fluid in spray form. A nasal support member 49 can be disposed around the dispensing head 40, to limit the insertion of said dispensing head 40 in a nostril and/or orient this insertion.

In the first embodiment of FIGS. 1 to 15, the dispensing head 40 is fixedly assembled on the main body 30, while in the second embodiment of FIGS. 16 to 29, the dispensing head 40 and the main body 30 are formed of one single one-piece part.

A central body 90 is provided in the main body 30, said central body 90 incorporating energy accumulation means, as will be described in more detail below. In the first embodiment of FIGS. 1 to 15, the central body 90 is made of a one-piece part with the central body 30, while in the second embodiment of FIGS. 16 to 29, the central body 90 is fixed to the central body 30.

The reservoir 10 is fixed in a support body 50, which is therefore secured to said reservoir 10, and which is displaced together with it.

The main body 30 comprises a lower side skirt 32 suitable for engaging with an actuating member 60. A finger-rest element 80 is formed on the main body 30 and/or on the dispensing head 40, or in a variant, it can be assembled on said main body 30 and/or on the dispensing head 40.

Said actuating member 60 is axially displaceable within said side skirt 32 of the main body 30 to perform successive actuations of the device. The actuating member 60 comprises at least one inclined tab 61 which is suitable for engaging with projections 51, 52 of the support body 50 to perform successive actuations.

At least one return spring 70 is mounted between the actuating member 60 and the main body 30 and/or the dispensing head 40, to return said actuating member 60 in its starting position after each actuation. In the examples represented in the figures, there are two parallel springs 70, but other implementations with any number of springs are possible.

Operation of the device shown in the figures is as follows. In the rest position of FIGS. 1 and 16, the stopper piston 20 isolates the content of the reservoir 10 from the atmosphere. When the user simultaneously presses on the finger-rest 80 and on the actuating member 60, it will displace said actuating member 60 upwards within the side skirt 32 of the main body 30. This will push the support body 50 axially upwards, by way of the tabs 61 which will push on the shoulder 51 of said support body 50. This will compress the spring 70 and displace the reservoir 10 with respect to the main body 30. When the reservoir 10 starts to be displaced with respect to the main body 30, the piercing end 44 of the dispensing channel 43 will pierce the stopper piston 20 to make the inside of the reservoir 10 communicate with said expulsion channel 43. Continuing the actuation will cause a displacement of the piston 20 within the reservoir 10 and therefore a dispensing of the first dose. The fluid is thus pushed by said piston 20 through the perforator tip 44 and into the dispensing channel 43, then via the spray profile 39 and out of the device through the dispensing orifice 41.

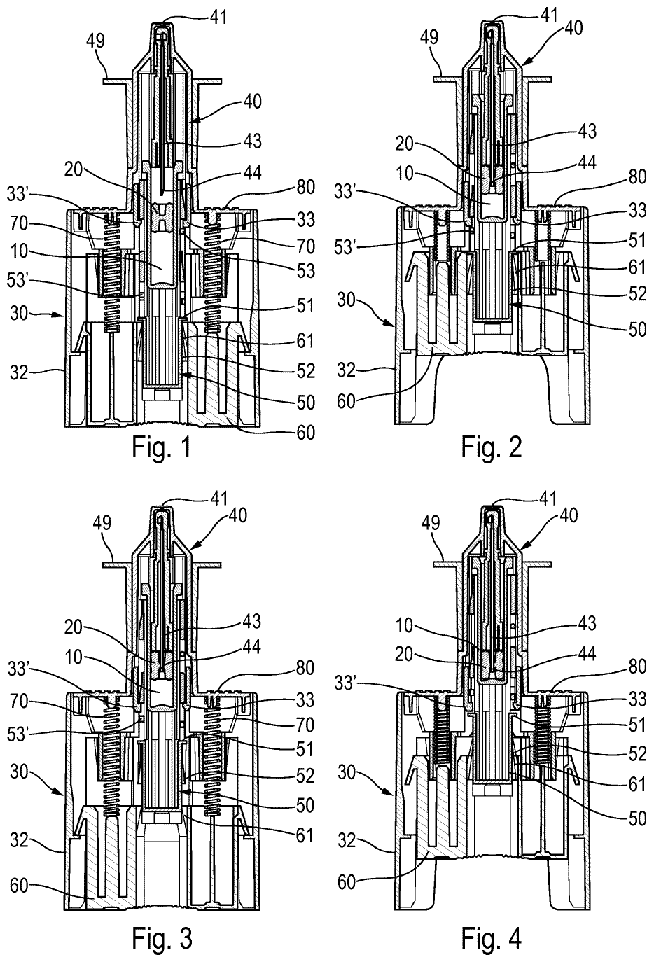

After dispensing of the first dose, the device is in the position represented in FIGS. 2 and 17, and when the user releases the actuating member 60, the spring 70 will return it to its starting position. During this return of the actuating member 60, the reservoir and the support body 50 do not return rearwards, as these two components remain held in the main body 30. Advantageously, anti-return means are provided to prevent a return of said support body 50 and/or of said reservoir 10. When the actuating member 60 returns under the effect of the return spring 70 to its rest position, the tabs 61 will be positioned under the second projection 52 of the support body 50, which will enable the user to actuate the device a second time to dispense the second dose of fluid product.

FIGS. 3, 4 and 18, 19 represent respectively the positions, before and after dispensing of the second dose.

The device comprises energy accumulation means. First energy accumulation means are provided for the first dose and second energy accumulation means are provided for the second dose.

In the first embodiment, represented in FIGS. 1 to 15, the central body 90 is made of a one-piece part with the central body 30, and the dispensing head 40 is fixed on said central body 30.

In the second embodiment represented in FIGS. 16 to 29, the dispensing head 40 is made of a one-piece part with the central body 30, and the central body 90 is fixed on said central body 30.

The first energy accumulation means comprise a first resistance element 53, formed on the support body 50, engaging with a corresponding first pushing element 33, 93 formed on the central body 90.

The second energy accumulation means comprise a second resistance element 53′, formed on the support body 50, engaging with a corresponding second pushing element 33′, 93′ formed on the central body 90.

The first and second resistance elements 53, 53′ are axially offset on the support body 50.

The first and second pushing elements 33, 33′, respectively 93, 93′, are disposed diametrically opposite on the central body 90.

The support member 50 comprises two diametrically opposite longitudinal profiles, each longitudinal profile comprising, starting with the upper axial edge: a first deformation profile 55, a first window 54, a second deformation profile 55′, substantially identical to the first deformation profile 55, and a second window 54′.

Each longitudinal profile only comprises one single resistance element 53, 53′, provided in only one of its windows 54, 54′. Thus, if a first longitudinal profile comprises the first resistance element 53 in its first window 54, then its second window has no resistance element. In this case, the other longitudinal profile comprises the second resistance element 53′ in its second window 54′, and its first window 54 has no resistance element.

The resistance elements 53, 53′ can be formed by breakable bridges, which are broken during actuation, or by deformable beams, which are deformed during actuation.

The first and second deformation profiles 55, 55′ of each longitudinal profile are advantageously identical, and each comprise: a central ramp 551, 551′ extending axially and inclined radially outwards towards the bottom, disposed between first and second axially straight side ridges and with a constant radial dimension 552, 552′ and 553, 553′. This can be seen, in particular, in FIG. 8.

The first and second pushing elements 33, 93 and 33′, 93′ are identical, the first pushing element 33, 93 engaging with a longitudinal profile and the second pushing element 33′, 93′ engaging with the other longitudinal profile.

The pushing elements being identical in the two embodiments, the description below will be made in reference to the first embodiment of FIGS. 1 to 15, with the pushing elements 33, 33′, but it is understood that this description applies in the same way as in the second embodiment of FIGS. 16 to 29, with the pushing elements 93, 93′.

Each pushing element 33, 33′ comprises a radially deformable tab 330, 330′, provided at its lower axial end with a central pushing zone 331, 331′, the axially lower surface of which comes into contact with a respective resistance element 53, 53′ during actuations.

Thus, to guarantee the integrity of these two pushing zones 331, 331′ during assembly, it is desirable that they undergo no impact during assembly. Likewise, to guarantee the integrity of the second pushing zone 331′ during the dispensing of the first dose, it is desirable that it undergoes no impact during this first dispensing. The first deformation profiles 55 engage with the two pushing elements 33, 33′ during assembly, and the second deformation profiles 55′ engage with the two pushing elements 33, 33′ during the dispensing of the first dose.

Each pushing zone 331, 331′ is disposed between a first and a second inclined zone 332, 332′ and 333, 333′, as can be seen in FIG. 7. Thus, the radially internal surface 335 of each pushing 33, element 33′ is advantageously T-shaped.

During assembly, the pushing zones 331, 331′ will each engage with a respective central ramp 551, while the inclined zones 332, 333 and 332′, 333′ will engage with the side ridges 552, 553.

Thus, as can be seen in FIGS. 10 to 15, which illustrate the assembly of the support member 50 in the main body 30, the inclined zones 332, 333 and 332′, 333′ of each pushing element 33, 33′ will slide over the upper axial edge of the side ridges 552, 553, which will deform the deformable tabs 330, 330′ radially outwards. Each pushing zone 331, 331′ will therefore avoid any contact with the support member 50, by first facing the part with the smallest diameter of each central ramp 551, then by moving away radially outwards according to the assembly.

At the end of assembly, which can be seen in FIG. 15, the deformable tabs 330, 330′ are snap-fitted in a respective first window 54, therefore with the first pushing element 33 disposed facing the first resistance element 53, which will make it possible to store energy during the dispensing of the first dose. The central pushing zone 331 having undergone no contact with the support member 50 during assembly, this energy storage will fully comply with the specifications provided.

With the second profiles 55′ being identical to the first profiles 55, the same applies during the dispensing of the first dose, such that after this first dispensing, the second pushing element 33′ is disposed facing the second resistance element 53′, which will make it possible to store energy during the dispensing of the second dose. The central pushing zone 331′ having undergone no contact with the support member 50, neither during assembly, nor during the dispensing of the first dose, this energy storage will comply fully with the specifications provided.

Naturally, the present invention has been described in reference to two embodiments, which are not limiting, and any useful modification can be applied to the present invention, without moving away from the scope of it, such as defined by the accompanying claims.

1. A device for dispensing a fluid product comprising a reservoir containing two doses of fluid product, a dispensing member, such as a piston, slidingly mounted in said reservoir to dispense a fluid product, a main body, a central body secured to said main body, a support member that accommodates said reservoir, a dispensing head provided with a dispensing orifice and an actuating member that is axially displaceable within said main body and engages with said support member to perform successive actuations of the device by displacing said support member with respect to said central body to thus displace said actuating member in said reservoir and thus dispense the fluid product through said dispensing orifice, a return spring being provided to return said actuating member into its starting position after each actuation, said device comprising energy accumulation means comprising first and second pushing elements formed on said central body and first and second resistance elements formed on said support member, said first pushing element being suitable for engaging with said first resistance element to accumulate energy during the dispensing of the first dose, and said second pushing element being suitable for engaging with said second resistance element to accumulate energy during the dispensing of the second dose, each pushing element comprising a radially deformable tab, provided at its lower axial end with a central pushing zone, the axially lower surface of which comes into contact with a respective resistance element during actuations, characterised in that:
said support member comprises two diametrically opposite longitudinal profiles, each longitudinal profile comprising, starting with the upper axial edge: a first deformation profile, a first window, a second deformation profile, and a second window,
the first window of a longitudinal profile comprises said first resistance element, and the second window of the other longitudinal profile comprises said second resistance element,
each deformation profile comprises: a central ramp extending axially and radially inclined outwards towards the bottom, disposed between first and second side ridges axially straight and with a constant radial dimension,
each central pushing zone is disposed between a first and a second inclined zone,
such that when said support member is axially displaced with respect to said central body during assembly, each pushing element engages with a respective first deformation profile, such that each central pushing zone does not come into contact with said support member.

2. The device according to claim 1, wherein when said support member is axially displaced with respect to said central body during the dispensing of the first dose, said second pushing element engages with a second deformation profile, such that its central pushing zone does not come into contact with said support member.

3. The device according to claim 1, wherein said first and second resistance elements are axially offset on said support body.

4. The device according to claim 1, wherein said first and second pushing elements are disposed diametrically opposite on said central body.

5. The device according to claim 1, wherein said resistance elements are formed by breakable bridges, which are broken during actuation, or by deformable beams, which are deformed during actuation.

6. The device according to claim 1, wherein said first and second pushing elements are identical, said first pushing element engaging with a longitudinal profile and said second pushing element engaging with the other longitudinal profile.

7. The device according to claim 1, wherein said first deformation profiles engage with the two pushing elements during assembly, and said second deformation profiles engage with said two pushing elements during the dispensing of the first dose.

8. The device according to claim 1, wherein the radially internal surface of each pushing element is T-shaped.

9. The device according to claim 1, wherein, during assembly, said pushing zones will each engage with a respective central ramp of a first deformation profile, while said inclined zones will engage with said side ridges.

10. Device according to claim 1, wherein, during the dispensing of the first dose, said pushing zones will each engage with a respective central ramp of a second deformation profile, while said inclined zones will engage with said side ridges.

11. The device according to claim 1, wherein, during assembly, said inclined zones of each pushing element slide over the upper axial edge of said side ridges of said first deformation profile, which deforms said deformable tabs radially outwards, such that each pushing zone avoids any contact with said support member, by first facing the part with the smallest diameter of each central ramp, then by moving away radially outwards according to the assembly.

12. The device according to claim 1, wherein, during the dispensing of the first dose, said inclined zones of each pushing element slide over the upper axial edge of said side ridges of said second deformation profile, which deforms said deformable tabs radially outwards, such that each pushing zone avoids any contact with said support member, by first facing the part with the smallest diameter of each central ramp, then by moving away radially outwards according to the dispensing of the first dose.

13. The device according to claim 1, wherein, at the end of assembly, said deformable tabs are snap-fitted in a respective first window, said first pushing element being disposed facing said first resistance element, to store energy during the dispensing of the first dose.

14. The device according to claim 1, wherein, at the end of dispensing of the first dose, said deformable tabs are snap-fitted in a respective second window, said second pushing element being disposed facing said second resistance element, to store energy during the dispensing of the second dose.
